(19) United States
(12) Patent Application Publication
(10) Pub. No.: US 2008/0254049 A1
(43) Pub. Date: Oct. 16, 2008

(54) Composition for Treatment of Colds and Associated Symptoms

(75) Inventors: Erin TARABORRELLI, Oakville (CA); Marvin HEUER, Oakville (CA); Ken CLEMENT, Oakville (CA); Megan THOMAS, Oakville (CA); Shan CHAUDHURI, Brampton (CA)

Correspondence Address: IOVATE HEALTH SCIENCE RESEARCH INC., 381 North Service Road West, Oakville, ON, L6M 0H4, CA

(73) Assignee: Iomedix Cold International SRL, Belleville (BB)

(21) Appl. No.: 12/099,937

(22) Filed: Apr. 9, 2008

Related U.S. Application Data
(60) Provisional application No. 60/911,005, filed on Apr. 10, 2007.

(51) Int. Cl. A61K 39/00; A61P 31/16

(52) U.S. Cl. 424/184.1

(57) ABSTRACT

A homeopathically designed composition comprising zinc and dilute amounts of Allium cepa and Aconite napellus for the alleviation of symptoms of a cold is provided. The composition may further comprise Antimonium crudum, Bryonia alba and Gelsemium.

### RELATED APPLICATIONS

The present application is related to and claims benefit of priority to U.S. Provisional Patent Application Ser. No. 60/911,005, entitled “Composition for Treatment of Colds and Associated Symptoms,” filed Apr. 10, 2007, the disclosure of which is hereby fully incorporated by reference.

### FIELD OF THE INVENTION

The present invention is related to homeopathic compositions and methods for the alleviation of cold symptoms.

### BACKGROUND OF THE INVENTION

Immunity is a result of the adaptive immune response of mammals. In the case of an adaptive immune response, the body has a “memory” for previously encountered antigens such that a second, more vigorous response is mounted by the body upon a second exposure to the same antigen.

In conventional medicine, the normal functioning of the adaptive immune response of mammals is taken advantage of in that vaccines are routinely administered for diseases such as measles, rubella, influenza, and recently cervical cancer. Specifically, denatured antigens and substances known to active the innate immune system, adjuvants, are administered to a mammal. The denatured antigens are essentially harmless to the mammal, yet activate the adaptive immune system to produce antibodies, or immunogens, which can neutralize proteins similar to the denatured antigens given. After this, when the body encounters a virus or other pathogen having a similar make-up, the body can quickly produce antibodies against the invader due to the adaptive immiune system.

This system of “vaccination” is also employed in allergy immunotherapy, wherein a treatment consisting of small doses of allergens is injected into a mammal, resulting in the alleviation of allergy symptoms as a result of an immune response. This method of treatment of is particularly useful in case of treating an individual against inhaled allergen, and anaphlaxis.

Since the antigens, or proteins, which are administered to an individual, elicit an immune response in the body to confer future immunity against the specific antigen, they are referred to as therapeutic proteins. This concept is the basis for homeopathic medicine. A substance which elicits a very similar symptomatic response is administered to an individual at a low dose; enough to active the adaptive immune system. For example, if the symptoms that a patient is expressing are similar to the symptoms expressed by a patient upon exposure to a particular pathogen, then the same pathogen is used a very low doses to treat the patient. The pathogen upon administration induces the body to produce immunogens which combat the symptoms expressed, and thus providing a remedy.

In homeopathic medicine, the pathogen is generally administered in an aqueous solution at a very low dose. Traditionally, amounts of substances present in a homeopathic remedy are often referred to as its dilution factor wherein a 1:10 dilution is equal to 1× (× potencies). For example, a 10× dilution is a dilution factor of 10¹⁰. Therefore the actual amount of antigen administered is very small. As such, it is understood that in the dilution, an essential property of the substance that was diluted is retained in the aqueous medium; enough to elicit a response of the adaptive immune system in a manner similar to a conventional vaccine.

Remedies given in homeopathic medicine are generally given as oral compositions. They are often placed on or under the tongue. By administering in this way, then antigenic proteins are not destroyed by gastric and intestinal juices, thus allowing them to reach lymphoid tissues intact. Generally, homeopathic treatments are administered orally, sublingually, nasally, ophthalmically and topically. In these routes of administration, chemical degradation of the antigen most likely avoided.

### SUMMARY OF THE INVENTION

The present invention is directed towards a homeopathically designed composition comprising zinc and dilute amounts of Allium cepa and Aconite napellus for the alleviation of symptoms of a cold. In an aspect of the present invention, the Allium cepa and Aconite napellus are present in dilute amounts able to induce the body of a mammal to produce immunogens able to alleviate the symptoms of a cold. In a further aspect of the invention, the composition may be administered to the mammal orally.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

As used herein, the term ‘cold’ is defined as an acute and contagious infection of the upper respiratory tract. A cold for the purposes of the present invention expresses rhinitis a symptom as well as sneezing, a stuffy feeling in the head, slight headache, watery eyes, general aching and listlessness, an inability to concentrate, coughing, headache, hoarseness (scratchy throat), nasal congestion, nasal discharge, and sore throat. A cold also, on occasion presents with a fever.

The present invention is directed towards a homeopathically designed composition comprising zinc and dilute amounts of Allium cepa and Aconite napellus for the alleviation of symptoms of a cold. An aspect of the present invention comprises at least zinc and homeopathically acceptable amounts of Allium cepa and homeopathically acceptable amounts of Aconite napellus.

According to various aspects, the present invention may further comprise combinations of Antimonium crudum, Bryonia alba and Gelsemium.

Zinc

Zinc is an essential mineral found in most cells of the body where it functions as a cofactor for numerous enzymes. As such, zinc plays an important role in many diverse functions. However, one of the most common claims for the use of supplemental zinc is for enhanced immune function.

Zinc has been shown to reduce the duration of common colds. The mechanism by which zinc may enhance the immune system has been largely elucidated and is multifaceted but involves modulation of the expression of cytokines. Additionally, zinc is co-factor for upwards of 70 enzymes in the body, such as superoxide dismutase, and it is understood that ensuring an adequate supply of this co-factor aids in the alleviation of the common cold.

It is herein understood by the inventors that zinc will reduce the duration of cold symptoms. It is further understood that this is due, at least in part, to the activity of zinc modulating the expression of immune signaling molecules and acting as an enzymatic co-factor.

In an embodiment of the present invention, which is set forth in greater detail in the example below, the homeopathic composition comprises a source of zinc. A serving of the homeopathic composition comprises from about 5 mg to about 25 mg of a source of zinc. The preferred dosage of a serving of the homeopathic composition comprises about 15 mg of a source of zinc.

Allium cepa

Allium cepa is the common onion and has been used in traditional and homeopathic medicine to treat many diseases involving immune modulation including the common cold. Quercetin, a flavonoid, is found in onions and is reported to have anti-allergic, antioxidant, antiviral, anti-inflammatory and cardiovascular-protective activity. Quercetin has been shown to suppress several proinflammatory and allergy signaling molecules in human mast cells which likely contributes to alleviating the symptoms of cold infection. In homeopathic medicine Allium cepa is indicated to treat cold-related symptoms of the nose and eyes.

It is herein understood by the inventors that Allium cepa will treat cold-related symptoms of the nose and eyes. Allium cepa has long been shown to cause sneezing and itchy red eyes, when administered in large doses. However, in homeopathic medicine, substances known to elicit adverse effects in large does are administered in highly dilute forms to induce the body to produce immunogens able to alleviate the symptoms of an allergy. Therefore, it is herein understood by the inventors that a highly dilute form of Allium cepa will facilitate the alleviation of cold symptoms, mainly sneezing and itchy red eyes.

In an embodiment of the present invention, which is set forth in greater detail in the example below, the homeopathic composition comprises Allium cepa. A serving of the homeopathic composition comprises from about 2× to about 6× of Allium cepa. The preferred dosage of a serving of the homeopathic composition comprises about 4× of Allium cepa.

Aconite napellus

Aconite napellus is obtained from the herb commonly known as Monkshood and is used in homeopathic medicine to assist the natural immune system and treat sneezing and itching eyes.

It is herein understood by the inventors that Aconite napellus will assist the natural immune system in combating cold-virus infection and treat the cold-related symptoms of sneezing and itching eyes.

In an embodiment of the present invention, which is set forth in greater detail in the example below, the homeopathic composition comprises Aconite napellus. A serving of the homeopathic composition comprises from about 4× to about 8× of Aconite napellus. The preferred dilution of a serving of the homeopathic composition comprises about 6× of Aconite napellus.

Antimonium crudum

Antimonium crudum, also known as antimony sulfide, is used in homeopathic remedies to treat dry mucous membranes such as the lips and nose as would occur during the course of a cold infection.

It is herein understood by the inventors that Antimonium crudum will treat cold-related symptoms of dry mucous membranes such as the lips and nose. Such symptoms may be the direct result of infection or the effects of irritation from excessive sneezing, running and wiping.

In an embodiment of the present invention, which is set forth in greater detail in the example below, the homeopathic composition comprises Antimonium crudum. A serving of the homeopathic composition comprises from about 5× to about 15× of Antimonium crudum. The preferred dilution of a serving of the homeopathic composition comprises about 10× of Antimonium crudum.

Bryonia alba

In homeopathic medicine, Bryonia alba is used to treat muscle aches, headaches and cough.

It is herein understood by the inventors that Bryonia alba will treat cold-related symptoms of muscle aches, headaches and cough.

In an embodiment of the present invention, which is set forth in greater detail in the example below, the homeopathic composition comprises Bryonia alba. A serving of the homeopathic composition comprises from about 2× to about 6× of Bryonia alba. The preferred dilution of a serving of the homeopathic composition comprises about 4× of Bryonia alba.

Gelsemium

Gelsemium is a genus of flowering plant. Constituents of Gelsemium have been shown to have analgesic and anti-inflammatory effects. In homeopathic remedies, Gelsemium is indicated in the treatment of sore throat and sneezing.

It is herein understood by the inventors that Gelsemium will treat cold-related symptoms of sore throat and sneezing. This is due, at least in part, to the ability of Gelsemium to confer analgesic and anti-inflammatory effects.

In an embodiment of the present invention, which is set forth in greater detail in the example below, the homeopathic composition comprises Gelsemium. A serving of the homeopathic composition comprises from about 3× to about 12× of Gelsemium. The preferred dilution of a serving of the homeopathic composition comprises about 6× of Gelsemium.

In a preferred embodiment of the present invention, the composition is comprised of a homeopathically designed composition comprising zinc and dilute amounts of Allium cepa and Aconite napellus for the alleviation of symptoms of a cold.

Not wishing to be bound by theory, it is believed that the components of the present invention will act in accordance to the theory of homeopathic medicine to substantially and simultaneously stimulate and support the immune system of an individual to relieve cold symptoms and lesson the likelihood of cold infection.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a powder or tablet suitable for dilution in water in accordance with the preparation and administration of homeopathic formulas.

Furthermore, the dosage form of the homeopathic composition may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art may be utilized in methods to relieve cold symptoms and lesson the likelihood of cold infection in an individual.

Although the following example illustrates the practice of the present invention in one of its embodiments, the example should not be construed as limiting the scope of the invention. Other embodiments will be apparent to one of skill in the art from consideration of the specifications and example.

### EXAMPLE

A homeopathic composition, for the alleviation of cold symptoms, comprising the following ingredients, in homeopathically acceptable levels of dilution, per serving is prepared for consumption once daily from a tablet, by dissolving in water:
About 15 mg of Zinc, Allium cepa, Aconite napellus, Antimonium crudum, Bryonia alba and Gelsemium.

Extensions and Alternatives

In the foregoing specification, the invention has been described with a specific embodiment thereof, however, it will be evident that various modifications and changes may be made thereto without departing from the broader spirit and scope of the invention.

What is claimed:

1. An orally administrable therapeutic composition comprising an effective amount of zinc and a diluted amount of at least two immunogens; wherein said immunogens are Allium cepa and Aconite napellus, and the immunogens having immunotherapeutic effects against the symptoms of a cold in a warm blood animal.

2. The composition of claim 1 wherein the zinc modulates the expression of immune signaling molecules.

3. The composition of claim 2 wherein the modulation the expression of immune signaling molecules effectively alleviates inflammation-related symptoms of a cold in a mammal.

4. The composition of claim 1 wherein the Allium cepa and Aconite napellus are present in homeopathically acceptable amounts.

5. The composition of claim 4 wherein the homeopathically acceptable amounts of Allium cepa and Aconite napellus are able to induce the body of a mammal to produce immunogens able to alleviate the symptoms of a cold.
